(19) United States
(12) Patent Application Publication
(10) Pub. No.: US 2022/0031310 A1
(43) Pub. Date: Feb. 3, 2022

(54) Biometallic Alloy Surgical Staples and Methods

(71) Applicant: MagStape LLC, Sarasota, FL (US)

(72) Inventors: Maurice De La Rosa, Santa Rosa, CA (US); Mark Paquin, Nokomis, FL (US)

(73) Assignee: MagStape LLC, Sarasota, FL (US)

(21) Appl. No.: 17/388,757

(22) Filed: Jul. 29, 2021

Related U.S. Application Data
(60) Provisional application No. 63/057,908, filed on Jul. 29, 2020.

(51) Int. Cl. A61B 17/064; A61L 31/02; A61L 31/14

(52) U.S. Cl. CPC A61B 17/0644; A61B 2017/00004; A61L 31/148; A61L 31/022

(57) ABSTRACT

A surgical staple is provided. The surgical staple includes a crown having a center portion and a foldable portion, and a pair of legs extending orthogonally from the crown and configured to puncture tissue. The crown and the pair of legs are fabricated from a biodegradable metal such that biodegradation causes the center portion to separate from the foldable portion and the pair of legs, and the foldable portion and the pair of legs are absorbable within the tissue.

### CROSS REFERENCE TO RELATED APPLICATION

The present application claims priority to and the benefit of U.S. Provisional Application Ser. No. 63/057,908, filed Jul. 29, 2020, which application is hereby incorporated by reference in its entirety.

### FIELD

The present disclosure relates to surgical stapling systems, and more particularly, to surgical stapling systems featuring staples fabricated from biodegradable and bioabsorbable alloy materials.

### BACKGROUND

Staples are frequently utilized for securing split skin, making staples superior to sutures. Surgical staples facilitate wound healing by apposing the wound edges. However, conventional metallic surgical staples are poorly tolerated and painful, require staple removal instruments, and tend to leave railroad track-like scarring. Biometallic staples are therefore more tolerable, do not require removal, and may leave less scarring.

In addition, the FDA issued guidance in March 2019 to surgical staple manufacturers to update labeling based on recent revelations about surgical stapler and staple safety. Current surgical staplers will continue to experience these safety issues regardless of label changes, and reclassifications will require manufacturers to re-test their products. Therefore, new surgical stapler technology that will meet the FDA reclassification requirements is desirable.

### SUMMARY

This Summary is provided to introduce a selection of concepts that are further described herein below in the Detailed Description. This Summary is not intended to identify key or essential features of the claimed subject matter, nor is it intended to be used as an aid in limiting the scope of the claimed subject matter.

According to one implementation of the present disclosure, a surgical staple is provided. The surgical staple includes a crown having a center portion and a foldable portion, and a pair of legs extending orthogonally from the crown and configured to puncture tissue. The crown and the pair of legs are fabricated from a biodegradable metal such that biodegradation causes the center portion to separate from the foldable portion and the pair of legs, and the foldable portion and the pair of legs are absorbable within the tissue.

According to another implementation of the present disclosure, a surgical staple system is provided. The system includes multiple surgical staples. Each of the surgical staples includes a crown having a center portion and a foldable portion, and a pair of legs extending orthogonally from the crown and configured to puncture tissue. The crown and the pair of legs are fabricated from a biodegradable metal such that biodegradation causes the center portion to separate from the foldable portion and the pair of legs, and the foldable portion and the pair of legs are absorbable within the tissue. The system further includes a binder coating configured to secure the surgical staples to each other into a single subassembly. The binder coating is selectively applied to a region of the crown.

According to still another implementation of the present disclosure, a method for manufacturing a surgical staple is provided. The method includes providing a sheet of biodegradable metal, chamfering the sheet to form at least one sharpened edge, and machining the sheet to form multiple surgical staples. Each of the surgical staples has a pair of pointed ends configured to puncture tissue. Each of the of surgical staples is positioned such that the pair of pointed ends includes the at least one sharpened edge.

### BRIEF DESCRIPTION OF THE DRAWINGS

The present disclosure is described with reference to the following Figures. The same numbers are used throughout the Figures to reference like features and like components.

FIG. 1A depicts an image of surgical staples being removed from a wound using known methods.

FIG. 1B depicts an image of scarring around the wound of FIG. 1A after the surgical staples have been removed.

FIG. 2A depicts a perspective view of a biometallic sheet that can be utilized to fabricate surgical staples using a laser cutting process according to an exemplary implementation of the present disclosure.

FIG. 2B depicts a side view of another biometallic sheet with chamfered edges that can be utilized to fabricate surgical staples using a laser cutting process.

FIG. 2C depicts a front view of the stages of fabricating staples from the biometallic sheet of FIG. 2B using a laser cutting process.

FIG. 3 depicts a front view of a surgical staple fabricated from the biometallic sheet depicted in FIG. 2A.

FIG. 4 depicts a perspective view of a surgical staple carrier which can be utilized with the surgical staples fabricated according to exemplary implementations of the present disclosure.

FIG. 5 depicts a perspective view of the surgical staple of FIG. 3 after it has been shaped by the carrier of FIG. 4.

FIG. 6A depicts a front view of the stages of forming an alternate geometry surgical staple according to another exemplary implementation of the present disclosure.

FIG. 6B depicts a front view of an alternate geometry surgical staple according to another exemplary implementation of the present disclosure.

FIG. 6C depicts a rear perspective view of the surgical staple of FIG. 6B.

FIG. 7A depicts a front view of an alternate geometry surgical staple according to another exemplary implementation of the present disclosure.

FIG. 7B depicts a front view of an alternate geometry surgical staple having a variable crown height according to another exemplary implementation of the present disclosure.

FIG. 7C is a side view of the surgical staple of FIG. 7B.

FIG. 8A depicts a wire-formed surgical staple having a constant circular cross-section.

FIG. 8B depicts a wire-formed surgical staple having a constant oval-shaped cross-section.

FIG. 8C depicts a laser cut surgical staple having a variable rectangular cross-section.

FIG. 9 depicts the detachment of a supracutaneous portion of a surgical staple from a subcutaneous portion.

FIG. 10A depicts a perspective view of an unsharpened wire-formed surgical staple.

FIG. 10B depicts a perspective view of the wire-formed surgical staple of FIG. 10A after a sharpening process has been performed.

FIG. 10C depicts a perspective view of a pre-sharpened laser cut surgical staple.

FIG. 10D depicts a perspective view of the surgical staple of FIG. 10C after an additional sharpening process has been performed.

FIG. 11A depicts a front view of a barbed surgical staple according to another exemplary implementation of the present disclosure.

FIG. 11B depicts the barbed surgical staple of FIG. 11A embedded in tissue at an incision site.

FIG. 12 depicts multiple laser cut surgical staples secured to each other using a binder coating and loaded on a carrier.

FIG. 13 depicts multiple wire-formed surgical staples loaded on a carrier.

FIG. 14 depicts the detachment of a supracutaneous portion of the surgical staple including binder coating from a subcutaneous portion.

FIG. 15 depicts a front view of an alternate geometry surgical staple having inverted tip geometry according to another exemplary implementation of the present disclosure.

FIG. 16 depicts a front view of an alternate geometry surgical staple having interlocking tip geometry according to another exemplary implementation of the present disclosure.

### DETAILED DESCRIPTION

In the present description, certain terms have been used for brevity, clarity, and understanding. No unnecessary limitations are to be inferred therefrom beyond the requirement of the prior art because such terms are used for descriptive purposes only and are intended to be broadly construed.

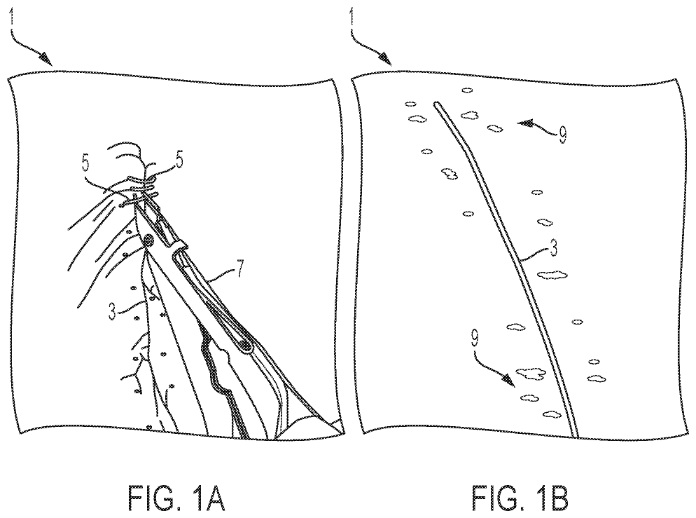

FIG. 1 depicts an area of tissue 1 having a wound 3 that is closed utilizing prior art surgical staples 5. After the surgical staples 5 are removed using a tool 7, significant scarring remains both at the site of the wound 3, as well as the sides 9 of the wound 3 that were punctured by the staples.

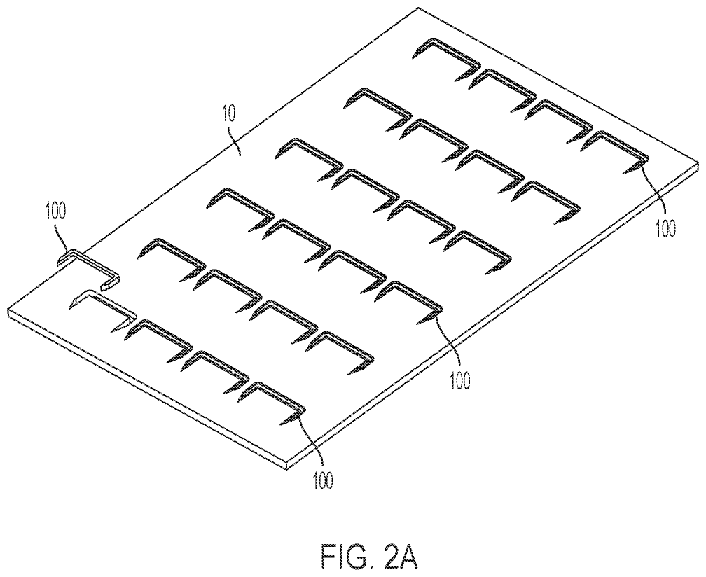

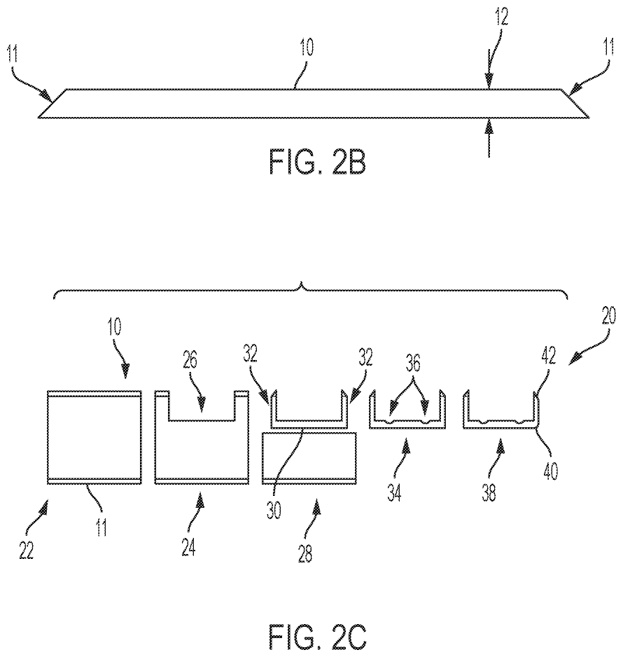

Turning now to FIGS. 2A-2C, aspects of an improved surgical staple system including laser cut staples are depicted. FIG. 2A depicts an exemplary sheet 10 from which multiple staples 100 may be cut. As used herein, the “sheet” 10 may include any raw material having a thickness (e.g., thickness 12, depicted in FIG. 2B) that is substantially less than its length or width. For example, the thickness may be a third or less than the length or width (e.g., raw material in strip form that is 3 mm wide and 1 mm thick). Accordingly, in various embodiments, the sheet 10 may include raw material in the form of a rectangular sheet, a square sheet, a strip, or a ribbon, such as produced from either slabs of pressed or rolled metal or wire drawn through dies.

Laser-cutting has advantages over other methods of surgical staple manufacturing, such as wire-forming, because laser-cutting from sheets of metal is a 2D technology that provides high-precision, tight tolerances, flexibility, repeatability, speed, cost-effectiveness, high quality, contactless cutting, versatility, and automation to produce a staple with a square or rectangular-shaped cross section. A laser-cut surgical staple with a square or rectangular cross section is more stackable in the staple carrier and may prevent staple jamming and misfiring.

In various embodiments, the surgical staple 100 may be made from a fully-absorbable biometallic alloy. In some embodiments, the biometallic alloy is a magnesium-alloy (MA), chemically composed of magnesium, zinc, and rare earth elements (REE). The MA with REE is biocompatible, has high-tensile strength, and good break elongation, and was initially developed to be drawn into wire or a tubular shape and fabricated via forming or laser-cutting into vascular stents.

In some embodiments, the surgical staple 100 may also be made from other biodegradable metals alloyed with alloying-metals comprising a combination of elements such as alkali and alkaline earth metals, select transition metals and REE, including calcium, dysprosium, iron, lithium, magnesium, manganese, neodynium, a lanthanide, yttrium, zirconium, and zinc.

In some embodiments, the one or more alloying (i.e., non-magnesium) elements comprise up to 35% of the total biodegradable metal weight. In another embodiment, the alloying elements are less than 5% of the total biodegradable metal weight and comprise lithium, zinc, calcium, and manganese. In other examples, the alloying elements may comprise up to 10%, up to 20%, or up to 30% of the total biodegradable metal weight. In one embodiment where the alloying elements comprise up to 12% of the total biodegradable metal weight, at least 10% is comprised of REE, such as dysprosium, and the remaining 2% is comprised of a different alloying element, such as zinc, neodynium, and zirconium.

Using different biometals and a combination of biometallic alloys can influence the degradation and mechanical behavior of the resulting surgical staple. It may be used in various applications, such as internally (e.g., vessel anastomosis closure) or externally, as in topical wound closure. In certain embodiments, alloying magnesium with lithium, calcium, zinc, and manganese to produce an alloy that is both strong and ductile, and free of REE, making the alloy suitable for implantation inside the body. Alloys free of REE may degrade faster compared to REE containing alloys which may be easier for body to metabolize the MA free of REE. Pathology data of alloys free of REE and those containing REE, indicate a better tolerability towards alloys free of REE when compared to alloys with rare elements. This is visible as fully degraded alloys free of REE appear as empty spaces in histomorphometry by image analysis whereas alloys with REE that are being degraded appear as more eosinophilic. These results indicate that the presence of REE delayed the dissolution of the alloy.

Rare earth-containing alloys may degrade at a slower rate when compared to non-rare earth containing alloys. A slower degradation may be better suited in certain applications, such as skin stapling.

Turning now to FIG. 2B, a side view of the biometallic sheet 10 is depicted. In an exemplary embodiment, the biometallic sheet 10 includes one or more chamfered or filleted edges 11. The chamfered edges 11 may be formed on the sheet using any suitable process, for example, machining, grinding, stamping, or drawing. As described in further detail below, the chamfered edges 11 may act to “pre-sharpen” the tips of the staples. In an exemplary implementation, the sheet 10 has a thickness 12 of approximately 1 mm, however in some embodiments may have a thickness ranging from 0.25 to 1.5 mm. As depicted in FIG. 2A, multiple staples 100 may be cut from a single sheet 10 of biometal to the loadable shape.

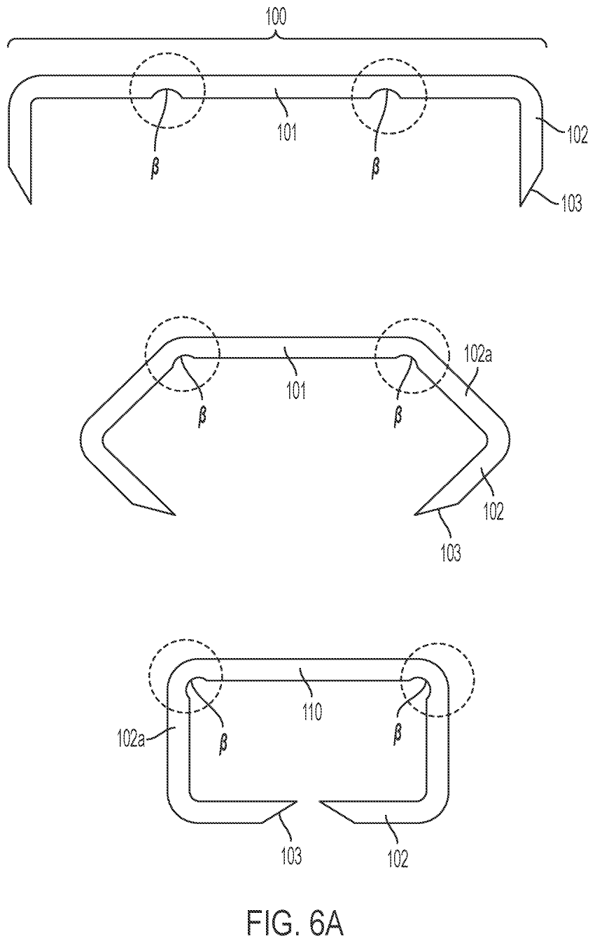

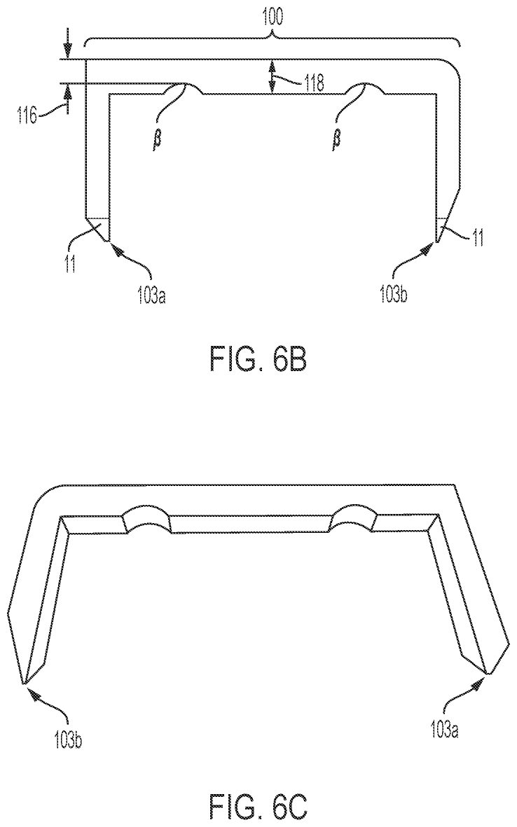

FIG. 2C depicts a process 20 for fabricating pre-sharpened laser cut staples from a single sheet 10 of biometal. As shown in step 22, the sheet 10 with chamfered edges 11 is provided. At step 24, a rectangular cut 26 is made along one of the chamfered edges 11. At step 28, a horizontal cut 30 is made to form a crown of the surgical staple, and regions 32 are removed to form the staple legs. At step 34, relief regions 36 (see further details with respect to FIGS. 6A-6C below) may be cut in the crown. At step 38, additional finishing operations may be performed to round corners 40 of the crown, and/or further sharpen the tips 42 of the legs. In some embodiments, one or more of the steps 22-38 may be combined in a single step of a laser cutting process. In still further embodiments, one or more of the steps in the process 20 may be performed using a different machining process than laser cutting, for example, photochemical etching or stamping or by combining processes, such as stamping to create coarser features and then laser cutting to create finer detailed features (e.g., relief regions 36, sharpened tips 42).

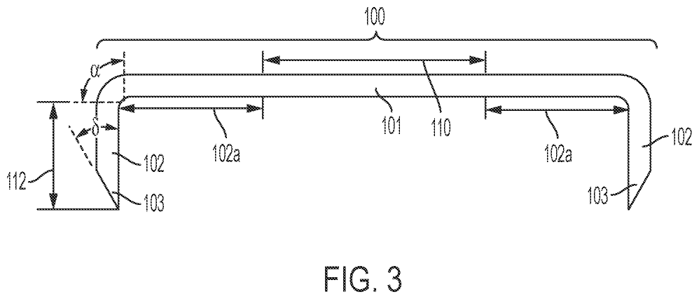

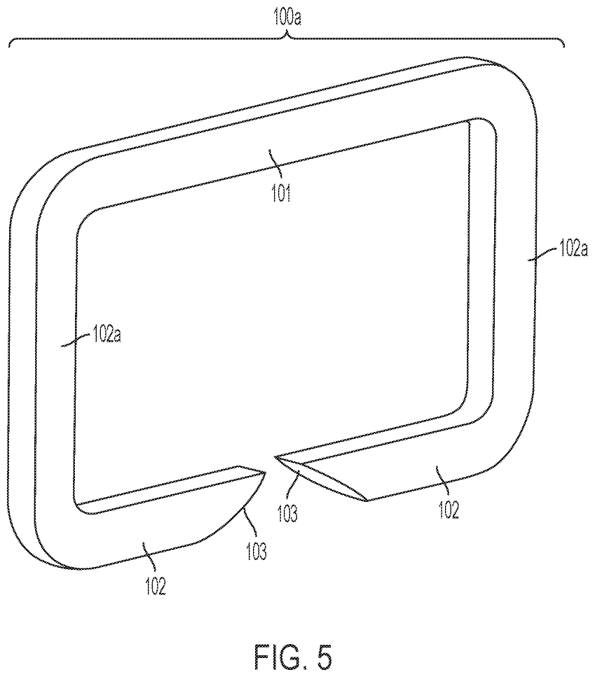

Referring now to FIG. 3, the finished laser cut staple 100 is depicted. As shown, the staple includes a crown 101 and a pair of legs 102 that extend generally perpendicularly from the crown 101. The crown 101 includes folding portions 102a and a center portion 110. When the staple is formed (as depicted and described with reference to FIG. 5 below), the folding portions 102a may be deformed to rotate inwardly relative to the center portion 110, which remains undeformed. In various implementations, the staple 100 may be used to close open skin wounds (e.g., surgical incisions, lacerations) by apposing the incised and lacerated edges of the skin or wound and securing each edge. In other implementations, the staple 100 may be used internally and left inside the body. When the staple is used to close open skin wounds, the folding portions 102a of the crown 101 may be driven subcutaneously into tissue, while the center portion 110 of the crown 101 is located supracutaneaously, that is, external to the body.

In various implementations, the crown 101 may measure within a range of 5-20 mm with a leg height 112 being preferably approximately 25% the length of the crown but within a range of 10-40%. The nominal bend angle α between the crown 101 and legs 102 is preferably 90° but may be within a range of 45°-135°. The nominal angle δ of the sharpened staple tips 103 is preferably 30° but may be within a range of 15°-40°. As depicted in FIG. 3, the height 114 of the cross section of the crown and legs can stay constant along the entire length of the surgical staple

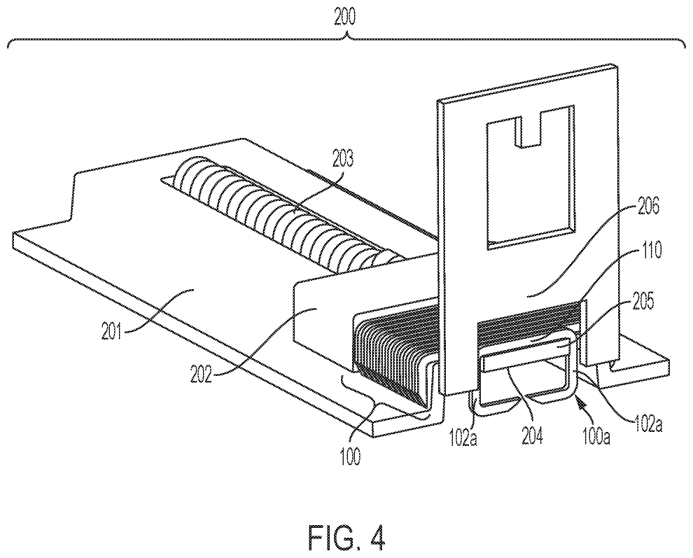

FIG. 4 depicts an exemplary carrier 200 which may be utilized to support the staples 100 as they are driven into and deformed for retention within tissue. The carrier 200 is shown to include a support track or frame 201 onto which surgical staples 100 are loaded. The carrier 200 further includes a pusher 202, a compression spring 203, a shaping hanger 204, crimping chamber 205, and crimping blade 206. In operation, the compression spring 23 applies pressure to the pusher 202, sliding the surgical staples 100 into the crimping chamber 205 with the crimping blade 206 positioned above the surgical staples 100. As the crimping blade is lowered onto each crown 101 of the surgical staples 100, the folding portions 102a are rotated inwardly, thus deforming the crown 101 and causing the surgical staple to be formed into its final shape 100a, as depicted in FIGS. 4 and 5.

In various embodiments, the laser-cutting pattern could be designed and shaped in such a way to facilitate the formation of the surgical staple to its final deployed shape. For example, in some embodiments as depicted in FIGS. 6A-6C, the shape of the deployed surgical staple 100 could be accomplished by thinning or reducing the height of the surgical staple cross section at certain places along its design lengths, thus causing the staple to preferentially bend at a thinned region. These reduced or thinned height regions, indicated by (3, may be generally arc-shaped. In an exemplary embodiment, the dimension 116 of the thinned surgical staple cross section can range between 50-95% of the surgical staple cross sectional height 118.

In various embodiments specifically depicted in FIGS. 6B and 6C, the laser-cut staple 100 could be cut from pre-shaped sheet of biometallic metal (e.g., sheet 10, depicted in FIGS. 2B and 2B) having chamfered edges 11, where the surgical staple tips 103 are cut to a trocar-like end with varying geometries 103a, 103b to facilitate post-laser cutting sharpening to a needle-like point tip.

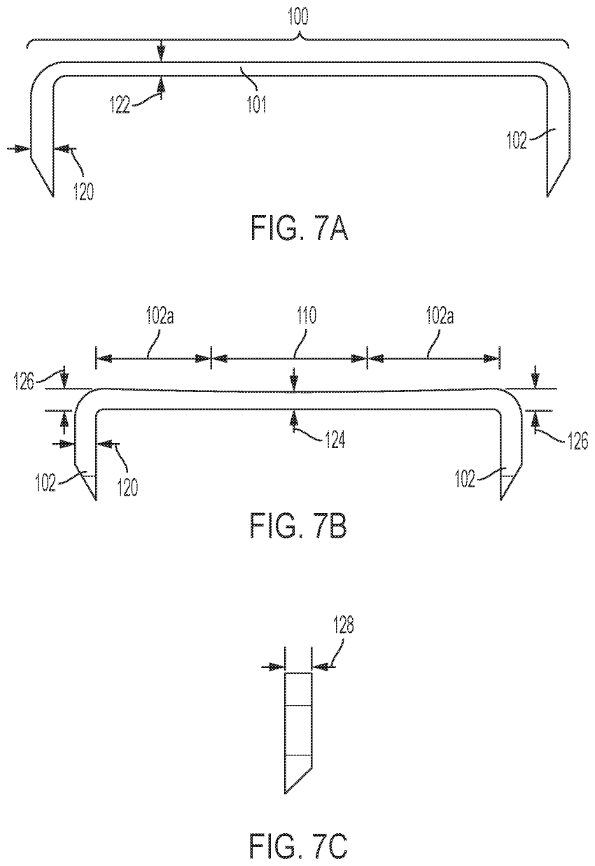

Referring now to FIGS. 7A-7C, in certain embodiments, the laser-cutting pattern could also be designed to vary the dimensions of the surgical staple to make certain regions or features stiffer. For example, FIG. 7A depicts a staple geometry in which a thickness 120 of the leg portion 102 has been broadened to stiffen the legs 102 relative to a height 122 of the crown 101 to permit the surgical staple 100 to more easily puncture tough tissue without deflecting undesirably. In various embodiments, the height 122 of the crown 101 may be 50-90% of the thickness 120 of the leg portion 102.

FIG. 7B depicts another staple having variable geometry. As shown, a height 124 of the center portion 110 of the crown may be smaller than the height 126 of the folding portions 102a. In this way, the folding portions 102a may more easily puncture tough tissue, and the crown may desirably bend at the junctures between the center portion 110 and the folding portions 102a. However, as depicted in 7C, although the heights of various portions of the crown 101 and the thickness of the legs 102 may be varied, the depth 128 may remain constant due to the use of a constant-thickness sheet (e.g., sheet 10, depicted in FIGS. 2B and 2C).

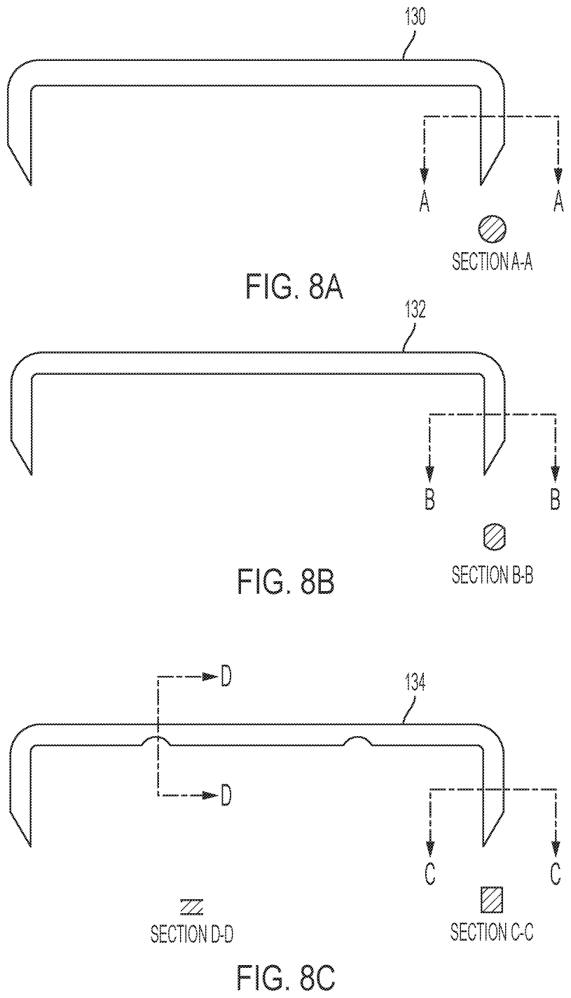

FIGS. 8A-8C are an illustration of the variety of cross sections possible between wire (FIGS. 8A and 8B) and laser-cut (FIG. 8C) surgical staples. The cross-sectional dimensions of the surgical staple 134 can be changed by manipulating the laser-cutting pattern, something that is not possible with the wire formed designs 130 and 132. By varying the cross-sectional dimensions, regions of the surgical staple can be made thinner (weaker) or broader (stiffer) as desired for the particular staple application.

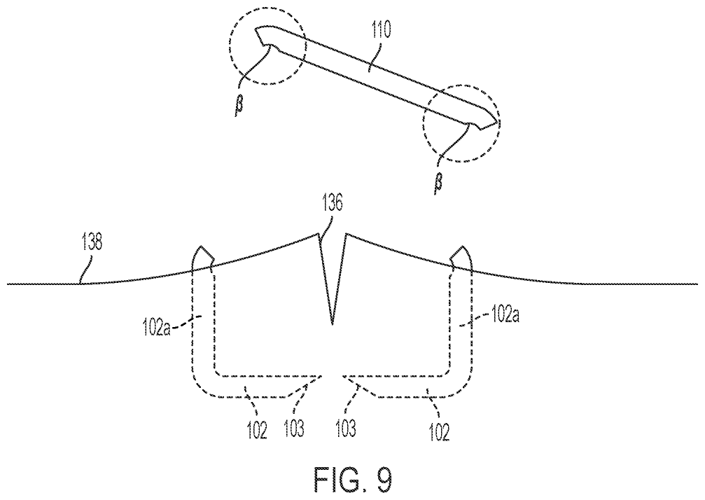

FIG. 9 is an illustration depicting an example of a surgical staple 100 after it has been driven into tissue 138 having an incision or wound 136. As shown, by varying cross-sectional dimensions, the thinner supra-cutaneous portion 13 loses mechanical integrity sooner via degradation and absorption into the tissue than the folding portions 102a and the legs 102, allowing the exposed center portion 110 of crown 101 to detach and fall away from the tissue 138.

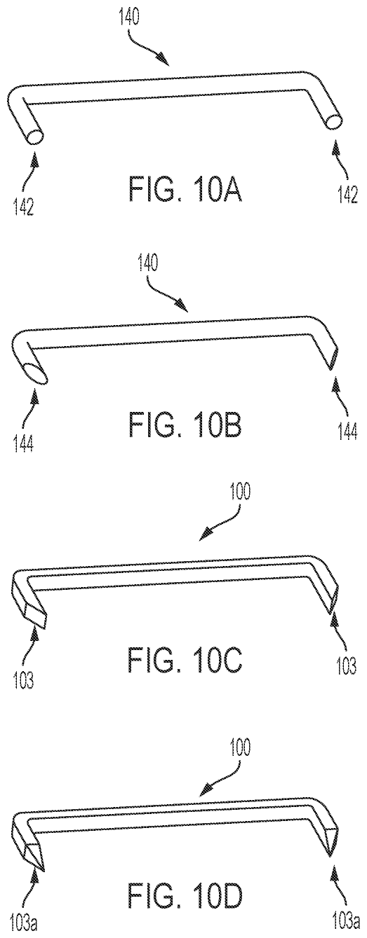

FIGS. 10A and 10B depict illustrations of a wire-formed surgical staple 140 that then must have its tips 142 sharpened into tips 144 via a secondary operation. This is in comparison to a laser-cut surgical staple 100 depicted in FIG. 10B with tips 103 pre-sharpened. With laser-cutting, no secondary sharpening operations are necessary, however FIG. 10D shows a laser-cut surgical staple with an additional secondary sharpening operation which results in trocar tips 103a. A trocar tip is a very efficient and effective tissue puncturing shape and so a trocar-tipped staple can be useful in facilitating operations such as small vessel anastomosis and puncturing the skin. By contrast, the wire staple 140 of FIG. 10B would need a tertiary step to result in a trocar tip. Thus, laser-cutting reduces the manufacturing steps necessary versus surgical staples made from wire which always require one or more operations to be sharpened.

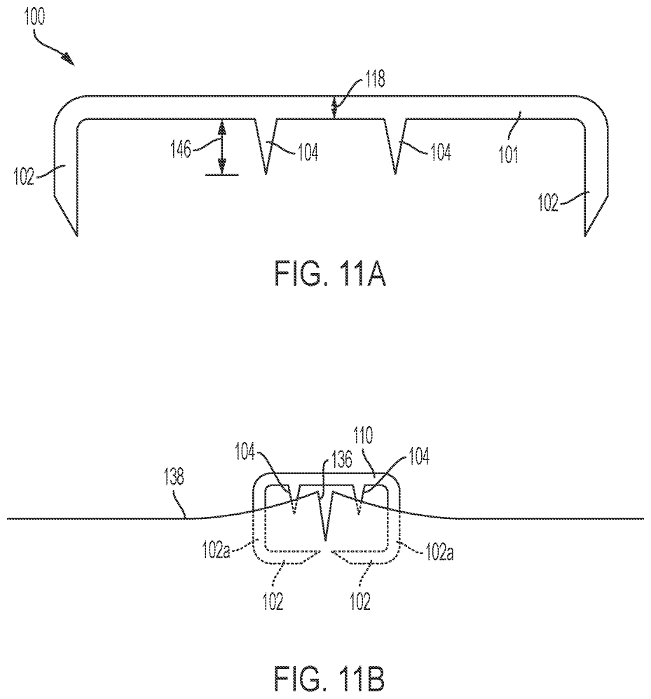

In particular embodiments, biometallic surgical staples produced with laser-cutting or photochemical etching methods can incorporate additional features that would not be achievable if the staples are made from wire or stamping processes. For example, as depicted in FIG. 11, the staple 100 may the addition of one or more barb features 104 that extend downwardly from the crown 101 to help secure and hold the tissue 138 together at the incision site 136 (see FIG. 11B). The barb features 104 can vary in height 146, preferably having a range of 10%-400% of the staple's nominal cross-sectional height 118.

In various embodiments, following laser-cutting, the surgical staples undergo surface cleaning and surface finishing operation to ensure the staples are biostable and residue-free.

Fabricating a surgical staple from a biodegradable metal, such as MA, may not only eliminate the need for a patient to have to return to a physician for removal, but could potentially minimize scarring in patients that scar more easily, especially when used in conjunction with a tissue adhesive.

In various embodiments, the biometallic surgical staple may be coated with a bioabsorbable coating to slow the corrosion and prolong the longevity and/or to modify the surface properties and/or to enhance the biocompatibility of the staple and/or to include a drug eluting component. In some embodiments, a biocompatible and biostable Parylene conformal coating is applied using chemical vapor deposition. The thickness of the Parylene coating is at least 1 micron. Parylene, a poly(p-xylylene) polymer, provides a moisture and a dielectric barrier.

In other embodiments, a conformal and flexible coating that uniformly covers the surfaces of the staple while adhering to the surfaces of the staple may also be used.

Conformal and flexible coatings of the type described herein, whether amorphous or crystalline in nature, may also improve the mechanical properties of the staple. This may be achieved by the combination of the biometallic properties (i.e., ductility) of the staple and those of the conformal, flexible coating, the latter being used to control both the bio-absorption profile and the re-coil of the metal. In particular embodiments, the conformal biodegradable polymer may include a plasticizer. In some embodiments, the conforming properties of the polymer may be optionally modified with the use of various biocompatible plasticizers.

In various embodiments, the conformal, flexible biodegradable polymer coating (with or without the addition of a plasticizer) may be applied evenly to the entire staple resulting in a coating with approximately the thickness of less than or equal to 1 micron covering the whole staple. In some embodiments, the coating thickness may be less than 10 microns to speed-up corrosion and subsequent degradation of the staple or more than 10 microns to extend the longevity of the biodegradable metal's mechanical properties.

The conformal biodegradable polymers suitable for this application include, but are not limited to, poly(L-lactide) or PLLA; poly(DL-Lactide) or PLA; Poly(L-lactide-co-D, L-lactide); polyglycolide or PGA; or poly(caprolactone), as well as their copolymers such as poly(lactide-co-glycolide). The biodegradable polymers may also include drug-eluting components, particularly for staples used in internal (i.e., inside the body) applications. In some embodiments, the biodegradable polymer may contain or may have coated thereon an antiproliferative drug-containing coating which may include a limus-based (e.g., rapamycin or its derivatives) drug or a taxane-based drug.

The conforming properties of the polymer may optionally be modified with the use of a biocompatible plasticizer. Suitable plasticizers include but are not limited to: alkyl citrates, such as triethyl citrate, acetyl triethyl citrate, tributyl citrate, acetyl tributyl citrate, or tri-(2ethylhexyl)citrate. Other suitable plasticizer may be cardanol, glycerol, polyethylene glycol.

In one embodiment, the conformal biodegradable polymer is PLLA or PLGA that is 70-99% by weight with acetyl tri-n-butyl citrate (ATBC) used as the plasticizer that is 1-30% by weight. In a preferred embodiment the biodegradable polymer is 85-95% by weight and the plasticizer 5-15% by weight. The addition of the plasticizer to the biodegradable polymer gives the conformal coating more elasticity, reducing the risk of coating delamination and tearing during cycling of the staple.

In particular embodiments, the MA may be treated using an electrochemical process that creates a dense oxide layer. This process can apply an oxide layer of between 5 and 8 microns. However, in some embodiments, the dense oxide layer may exceed 8 microns in thickness or be less than 5 microns thick. In some embodiments, the surgical staple may also remain uncoated and rely on dimensional variations to alter and control the rate of degradation.

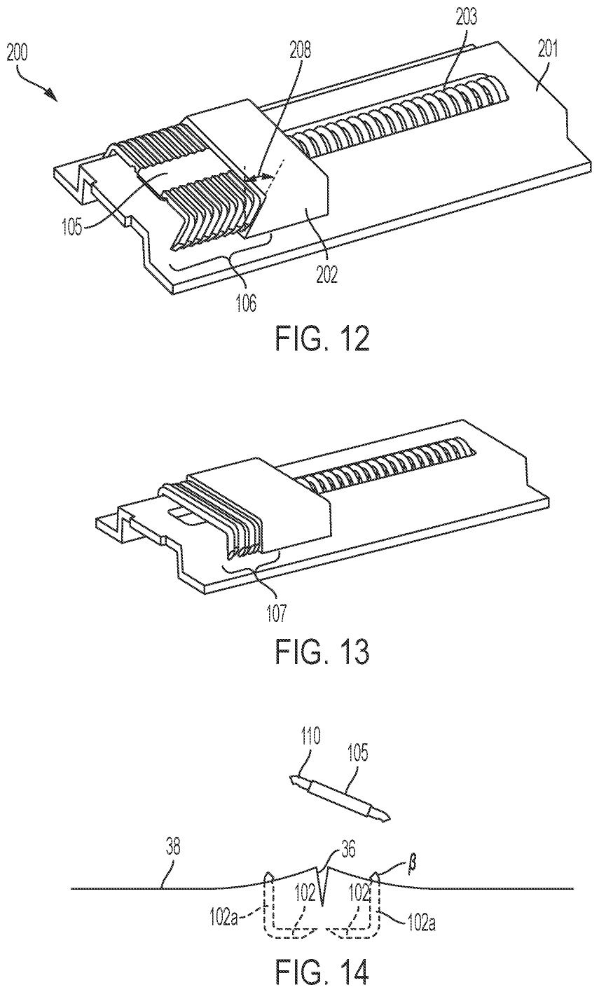

Referring now to FIG. 12, one method to facilitate the loading process of the surgical staples easier into the carrier instead of individually loading each surgical staple one-by-one is to apply a thin secondary coating 105 to a plurality of the surgical staples 100. The secondary coating 105 is applied by loading a plurality of the surgical staple one against the other onto a loading rack (not shown), such that the secondary coating binds the surgical staples together into a single, easier to handle subassembly 106. To avoid delaying the rate of degradation of the surgical staple from applying a secondary coating, the second coating 105 is selectively applied by masking-off areas where faster degradation is required (e.g., as depicted in FIG. 14, the folding portions 102a and the legs 102 that are embedded in tissue).

Therefore, the secondary coating is applied only to the outer surface area of the surgical staple 100, for example, the area of the staple that does not come into contact with the skin. The crown 101 of the staple will only degrade at the folding portions 102a of the crown, where the arc-shaped reliefs (3 in the corners are formed and where the staple enters the tissue and where the first layer of coating is applied. The center portion 110 of the crown may not fully degrade before falling off, and thus this area of the crown 101 that does not contact the skin becomes a logical area where a secondary layer of coating may be applied for the purposes of binding the staples 100 together without compromising the rate of degradation in other areas of the staple 100 where faster degradation is desired. The coating may be applied as a thin strip along the top of the stacked plurality of the surgical staple 100. The strip may be made from non-degradable or degradable polymers, such as a polylactide.

Binding the staples together may reduce the incidence for the stapler to jam by preventing surgical staples from riding over each other in the carrier/cartridge, as is depicted by staples 107 in FIG. 13. Surgical staples that bunch up and ride over each other in the carrier/cartridge are more prone to jam or misfire. The bunching and jamming of surgical staples in the carrier/cartridge can result from the constant spring force applied to the surgical staples from the metal spring, and/or from jostling due to shipping and handling, and/or from shifting of the surgical staples as they advance through the carrier/cartridge as the stapler is being used. Surgical staples cut from metal sheets have flat surfaces that are inherently better suited to stacking and packing against each other when compared to those made from round wire.

In various embodiments, the improved surgical staple carrier 200 depicted in FIGS. 4 and 12 comprises both metallic and non-metallic components. The non-metallic component comprises a structure 202 (or “pusher”) for pushing a plurality of surgical staples through the carrier before loading a single surgical staple into the crimping chamber. The pusher 202 can push against the surgical staples with a face perpendicular to the longitudinal axis of the compression spring 203, or at an angle 208, as is depicted in FIG. 12. Preferably, the angle 208 of the pusher face can range from 0-45°. Angling the pusher face while acting upon the surgical staples from pressure generated by the compression spring can cause the surgical staples to consistently ride up the angle and reliably bias a surface of the crown of the surgical staple against a surface of the carrier, thus reducing the likelihood of jamming during use of the stapler. The metallic components of the surgical staple carrier can comprise the support frame 201, the compression spring 203, the shaping hanger 204, and the crimping blade 206. In other embodiments, the staple support frame 201 and shaping hanger 204 can be fabricated from non-metallic components.

In some embodiments, the support frame 201, shaping hanger 204, and crimping blades 206 are covered by a protective layer of dielectric paint that can withstand sterilization, such as Cerablak®. The Cerablak® is a dense coating that is sprayed on to a thickness of at least one micron.

In particular embodiments, some metal components that are in direct contact with the innovative surgical staple may be made with injection molded plastic, or from a machined thermoplastic, such as a Delrin® (polyoxymethylene). The plastic may be selected based on its ability to tolerate sterilization. The plastic may also be selected based on the staple material, the application and location where the staple will be utilized, and how the staple material will be processed (e.g., worked annealed, and/or treated). Delrin® is used in making precision parts requiring low friction, high stiffness and dimensional stability. The thermoplastic can be used to fabricate the support frame and other components such as the shaping hanger 204 and crimping blades 206.

The use of dielectric paint and/or thermoplastic in the manufacture of the staple carrier insulates the carrier from the staples and acts to avoid the galvanic corrosion that can occur when two different metals contact each other in the presence of an electrolyte. Electrolytes are found in all bodily fluids, including blood, therefore management of corrosion risk is particularly important in medical applications.

In some embodiments, the MA surgical staple may recoil a nominal, yet functionally significant amount after retraction of the crimping blade 206 and ejection of the staple 100 from the carrier (e.g., carrier 400, see FIG. 4). In various embodiments, the stapler's crimping blade geometry is designed to over-deflect/shape the surgical staple to compensate for the biometal's inherent recoil so that the surgical staple springs back to the desired final shape.

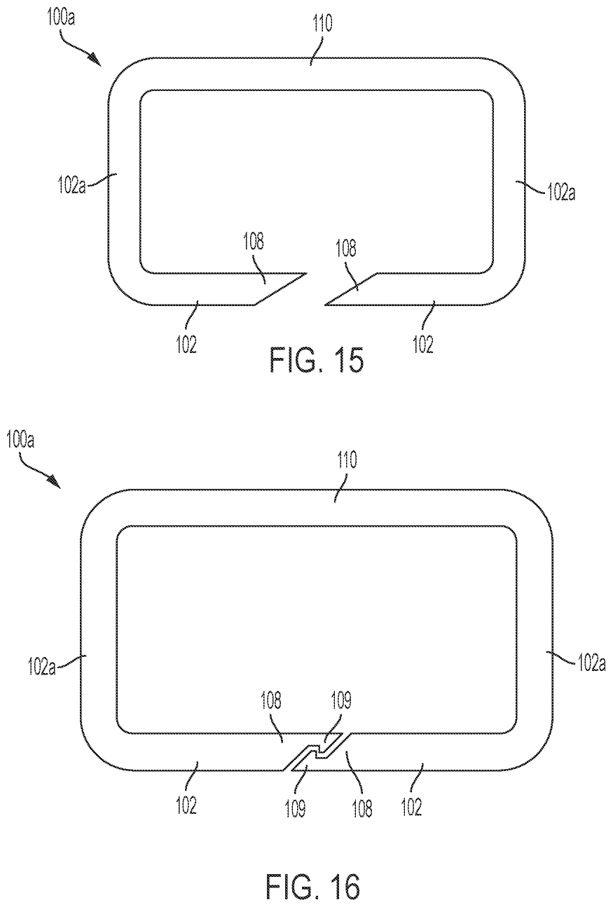

In particular embodiments, if the final shape of the surgical staple necessitates a small gap between the sharpened tips to be effective, it may be impossible to over-deflect the surgical staple without the tips contacting each other and impeding further deflection. As depicted in FIGS. 15 and 16 the tips 108 can be cut inverted relative to each other to overcome the issue of the tips touching. The staples depicted in FIGS. 15 and 16 may be particularly useful in interior applications (e.g., for securing tubular structures or blood vessels), and may be used in applications where ligation clips are currently utilized.

In some embodiments, the inverted tips 108 can overlap. Inverting the tips allows the surgical staple to over-deflect while in the crimping blade. The inverted tips 108 allow the tips to avoid hitting each other as the surgical staple is formed, thus permitting the MA surgical staple to spring back to the desired final shape and tip spacing to yield an effective design.

The method of deflecting the surgical staple described above may still leave the shaped surgical staple vulnerable to separation at the tips even if the surgical staple is initially formed to the desired shape. This is due to weaker mechanical properties of the MA when compared to more conventional metals used is the production of conventional, non-degradable metallic surgical staples. The weaker material properties of the MA could potentially allow an incision or wound to re-open. Re-opening of a wound can occur if the surgically stapled incision is in a location of high stress/tension on the body, which can lead to the separation of the incision during normal daily activities. The stress/tension may cause the tissue to separate/pull apart due to the MA lower tensile strength.

Therefore, in some embodiments and as depicted in FIG. 16, the tip geometry can be modified to not only facilitate over-deflection of the surgical staple by inverting the tip shape, but also by adding an interlocking feature 109 that allows the tips 108 to lock during the shaping and ejection phases of the surgical staple. The interlocking tip feature 108 is designed to prevent the tips from spreading apart, even if the incision is subject to high stress/tension.

### EXAMPLES

### Example 1

A surgical staple includes a crown having a center portion and a foldable portion, and a pair of legs extending orthogonally from the crown and configured to puncture tissue. The crown and the pair of legs are fabricated from a biodegradable metal such that biodegradation causes the center portion to separate from the foldable portion and the pair of legs, and the foldable portion and the pair of legs are absorbable within the tissue.

### Example 2

The surgical staple of Example 1, wherein the biodegradable metal comprises magnesium alloyed with at least one alloying element.

### Example 3

The surgical staple of Example 2, wherein the at least one alloying element is a rare earth element.

### Example 4

The surgical staple of Example 3, wherein the rare earth element comprises a lanthanide, yttrium, or zirconium.

### Example 5

The surgical staple of Example 2, wherein the at least one alloying element comprises lithium, calcium, zinc, or manganese.

### Example 6

The surgical staple of Example 2, wherein the at least one alloying element comprises up to 35% of the total biodegradable metal weight.

### Example 7

The surgical staple of Example 1, wherein the crown is tapered such that a minimum height region is located at the center portion and a pair of maximum height regions are located at the foldable portion proximate the pair of legs.

### Example 8

The surgical staple of Example 1, wherein the crown comprises a pair of arc-shaped relief regions configured to aid separation of center portion from the foldable portion.

### Example 9

The surgical staple of Example 1, wherein the surgical staple further comprises at least one barb extending from the crown between the pair of legs and configured to puncture the tis sue.

### Example 10

The surgical staple of Example 9, wherein the at least one barb comprises a pair of barbs configured to puncture the tissue on opposing sides of an incision or wound.

### Example 11

The surgical staple of Example 1, wherein the surgical staple is formed from a sheet of the biodegradable metal having a thickness ranging from 0.25 mm to 1.5 mm using a laser cutting process.

### Example 12

The surgical staple of Example 1, wherein each of the pair of legs terminates in a pointed end, and wherein the pointed ends are inverted relative to each other to compensate for material spring back during a crimping process.

### Example 13

The surgical staple of Example 1, wherein each of the pair of legs terminates in an interlocking tip and wherein the interlocking tips are configured to lock relative to each other during a crimping process.

### Example 14

The surgical staple of Example 1, wherein at least a portion of the surgical staple is coated with a biodegradable coating configured to provide a moisture and dielectric barrier, wherein the biodegradable coating has a thickness of at least 0.001 microns.

### Example 15

The surgical staple of Example 1, wherein at last a portion of the surgical staple is coated with an oxide layer having a thickness between 5 and 8 microns.

### Example 16

The surgical staple of Example 1, wherein the surgical staple is coated with a plasticized biodegradable polymer. The coating is applied by spraying layers and/or dip-coating layers onto the staple, creating a conformable and flexible coating, where the plasticized biodegradable polymer coating may be utilized to modulate the biodegradable metal bio-absorption profile when implanted into tissue, particularly inside the body. The biodegradable coating may, in certain embodiments, reduce or prevent polymer delamination, flaps, and cracking during the staple deformation/shaping process, which in turn will reduce inadvertent focal/localized and premature degradation that can impact the integrity of the biodegradable staple.

### Example 17

A surgical staple system includes multiple surgical staples. Each of the surgical staples includes a crown having a center portion and a foldable portion, and a pair of legs extending orthogonally from the crown and configured to puncture tissue. The crown and the pair of legs are fabricated from a biodegradable metal such that biodegradation causes the center portion to separate from the foldable portion and the pair of legs, and the foldable portion and the pair of legs are absorbable within the tissue. The system further includes a binder coating configured to secure the surgical staples to each other into a single subassembly. The binder coating is selectively applied to a region of the crown.

### Example 18

The surgical staple system of Example 17, wherein the crown comprises a pair of arc-shaped relief regions, and wherein the binder coating is applied between the pair of arc-shaped relief regions.

### Example 19

The surgical staple system of Example 17, further comprising a surgical staple carrier. The surgical staple carrier includes a support frame configured to receive the subassembly, a pusher coupled to a compression spring, the pusher and the compression spring configured to apply a force to the subassembly to slide the subassembly along the support frame, and a crimping blade configured to be lowered onto the crowns to drive each of the plurality of surgical staples into the tissue.

### Example 20

The surgical staple system of Example 19, wherein at least one of the support frame and the crimping blade are coated in a layer of dielectric paint having a thickness of at least 1 micron.

### Example 21

The surgical staple system of Example 19, wherein at least one of the support frame and the crimping blade are fabricated from a thermoplastic.

### Example 22

A method for manufacturing a surgical staple includes providing a sheet of biodegradable metal, chamfering the sheet to form at least one sharpened edge, and machining the sheet to form multiple surgical staples. Each of the surgical staples has a pair of pointed ends configured to puncture tissue. Each of the of surgical staples is positioned such that the pair of pointed ends includes the at least one chamfered edge.

### Example 23

The method of Example 22, wherein machining the sheet to form the plurality of surgical staples comprises forming a crown comprising a center portion and a foldable portion, and forming a pair of legs extending orthogonally from the crown and configured to terminate in the pair of pointed ends. The crown and the pair of legs are fabricated from a biodegradable metal such that biodegradation causes the center portion to separate from the foldable portion and the pair of legs, and the foldable portion and the pair of legs are absorbable within the tissue.

### Example 24

The method of Example 23, wherein machining the sheet to form the plurality of surgical staples further comprises forming a pair of arc-shaped relief regions configured to aid separation of the center portion from the foldable portion.

### Example 25

The method of Example 23, further comprising coating at least a portion of the plurality of surgical staples.

### Example 26

The method of Example 25, wherein coating at least a portion of the plurality of surgical staples comprises applying biodegradable coating configured to provide a moisture and dielectric barrier, and wherein the biodegradable coating has a thickness of at least 0.001 microns.

### Example 27

The method of Example 25, wherein coating at least a portion of the plurality of surgical staples comprises treating the plurality of surgical staples using an electrochemical process to form an oxide layer, and wherein the oxide layer has a thickness between 5 and 8 microns.

### Example 28

The method of Example 25, wherein coating at least a portion of the plurality of surgical staples comprises masking the pair of legs and the foldable portion of each of the plurality of surgical staples, applying a binder coating to the center portion of each of the plurality of surgical staples, the binder coating configured to secure the plurality of surgical staples to each other into a single subassembly.

### Example 29

The method of Example 22, wherein machining the sheet to form the plurality of surgical staples comprises a laser cutting process.

### Example 30

The method of Example 22, wherein machining the sheet to form the plurality of surgical staples comprises a stamping process.

### Example 31

The method of Example 22, wherein machining the sheet to form the plurality of surgical staples comprises a photochemical etching process.

### Example 32

The method of Example 22, wherein machining the sheet to form the plurality of surgical staples comprises a preliminary stamping process and a subsequent laser cutting process.

### Example 33

The method of Example 32, wherein the subsequent laser cutting process is configured to sharpen the pair of pointed ends.

### Example 34

The method of Example 32, wherein the subsequent laser cutting process is configured to cut at least one relief region in each of the plurality of surgical staples.

In the present disclosure, certain terms have been used for brevity, clarity, and understanding. No unnecessary limitations are to be implied therefrom beyond the requirement of the prior art because such terms are used for descriptive purposes only and are intended to be broadly construed. The different systems and methods described herein may be used alone or in combination with other systems and devices. Various equivalents, alternatives and modifications are possible within the scope of the appended claims.

What is claimed is:

1. A surgical staple, comprising:
a crown comprising a center portion and a foldable portion; and
a pair of legs extending orthogonally from the crown and configured to puncture tissue;
wherein the crown and the pair of legs are fabricated from a biodegradable metal such that biodegradation causes the center portion to separate from the foldable portion and the pair of legs, and wherein the foldable portion and the pair of legs are absorbable within the tissue.

2. The surgical staple of claim 1, wherein the biodegradable metal comprises magnesium alloyed with at least one alloying element.

3. The surgical staple of claim 2, wherein the at least one alloying element is a rare earth element.

4. The surgical staple of claim 3, wherein the rare earth element comprises a lanthanide, yttrium, or zirconium.

5. The surgical staple of claim 2, wherein the at least one alloying element comprises lithium, calcium, zinc, or manganese.

6. The surgical staple of claim 2, wherein the at least one alloying element comprises up to 35% of a total biodegradable metal weight.

7. The surgical staple of claim 1, wherein the crown is tapered such that a minimum height region is located at the center portion and a pair of maximum height regions are located at the foldable portion proximate the pair of legs.

8. The surgical staple of claim 1, wherein the crown comprises a pair of arc-shaped relief regions configured to aid separation of the center portion from the foldable portion.

9. The surgical staple of claim 1, wherein the surgical staple further comprises at least one barb extending from the crown between the pair of legs and configured to puncture the tissue.

10. The surgical staple of claim 9, wherein the at least one barb comprises a pair of barbs configured to puncture the tissue on opposing sides of an incision or wound.

11. The surgical staple of claim 1, wherein the surgical staple is formed from a sheet of the biodegradable metal having a thickness ranging from 0.25 mm to 1.5 mm using a laser cutting process.

12. The surgical staple of claim 1, wherein each of the pair of legs terminates in a pointed end, and wherein the pointed ends are inverted relative to each other to compensate for material spring back during a crimping process.

13. The surgical staple of claim 1, wherein each of the pair of legs terminates in an interlocking tip and wherein the interlocking tips are configured to lock relative to each other during a crimping process.

14. The surgical staple of claim 1, wherein at least a portion of the surgical staple is coated with a biodegradable coating configured to provide a moisture and dielectric barrier, wherein the biodegradable coating has a thickness of at least 0.001 microns.

15. The surgical staple of claim 1, wherein at least a portion of the surgical staple is coated with an oxide layer having a thickness between 5 and 8 microns.

16. The surgical staple of claim 1, wherein at least a portion of the surgical staple is coated with a plasticized biodegradable polymer.

17. A surgical staple system, comprising:
a plurality of surgical staples, each of the surgical staples comprising:
a crown comprising a center portion and a foldable portion; and
a pair of legs extending orthogonally from the crown and configured to puncture tissue;
wherein the crown and the pair of legs are fabricated from a biodegradable metal such that biodegradation causes the center portion to separate from the foldable portion and the pair of legs, and wherein the foldable portion and the pair of legs are absorbable within the tissue; and
a binder coating configured to secure the plurality of surgical staples to each other into a single subassembly, wherein the binder coating is selectively applied to a region of the crown.

18. The surgical staple system of claim 17, wherein the crown comprises a pair of arc-shaped relief regions, and wherein the binder coating is applied between the pair of arc-shaped relief regions.

19. The surgical staple system of claim 17, further comprising:
a surgical staple carrier, comprising:
a support frame configured to receive the subassembly;
a pusher coupled to a compression spring, the pusher and the compression spring configured to apply a force to the subassembly to slide the subassembly along the support frame; and
a crimping blade configured to be lowered onto the crowns to drive each of the plurality of surgical staples into the tissue.

20. The surgical staple system of claim 19, wherein at least one of the support frame and the crimping blade are at least one of:
coated in a layer of dielectric paint having a thickness of at least 1 micron; and
fabricated from a thermoplastic.
